(19) United States
(12) Patent Application Publication
(10) Pub. No.: US 2022/0133925 A1
(43) Pub. Date: May 5, 2022

(54) SANITIZING APPARATUS

(71) Applicants: WHOLETEK INC., Corunna (CA); ROGER H. WOODS LIMITED, Watford (CA)

(72) Inventors: Scott Gray, Corunna (CA); Douglas G. Pullman, Watford (CA)

(21) Appl. No.: 17/452,877

(22) Filed: Oct. 29, 2021

Related U.S. Application Data
(60) Provisional application No. 63/109,071, filed on Nov. 3, 2020.

(51) Int. Cl. A61L 2/10; A61L 2/26; A61L 2/24

(52) U.S. Cl. CPC A61L 2/10; A61L 2/26; A61L 2202/14; A61L 2202/11; A61L 2202/122; A61L 2/24

(57) ABSTRACT

A sanitizing apparatus for sanitizing one or more objects is provided. The sanitizing apparatus includes a housing defining an enclosure and one or more UV radiation sources including UV lamps disposed therein. The UV radiation sources are activated to provided sanitizing UV radiation to objects placed within the housing. The sanitizing apparatus further includes a temperature control device configured for maintaining the temperature of the UV lamps within an optimal temperature range to maximize UV radiation output over time.

### CROSS-REFERENCE TO RELATED APPLICATIONS

This application claims the benefit of prior Application No. 63/109,071, filed Nov. 3, 2020, which is incorporated by reference herein in its entirety.

### TECHNICAL FIELD

The present disclosure relates to the field of sanitizing systems, and in particular to a dual-purpose UV sanitizing apparatus for sanitizing objects such as wheelchairs, shopping carts, and other items, as well as having the ability to sanitize an air stream.

### SUMMARY

In an embodiment, a sanitizing apparatus is provided. The sanitizing apparatus includes a housing defining an enclosure; a UV radiation source arranged within the housing, including a UV lamp and configured to provide sterilizing UV radiation to the enclosure, and a temperature control device associated with the UV radiation source and configured to direct a flow of air into a near field ambient environment of the UV lamp to control the temperature of the UV lamp to within an optimal temperature range.

In a further embodiment, a method of sanitizing objects is provided. The method comprises placing an object to be sterilized in an enclosure defined by a housing of a sterilizing apparatus; activating a UV radiation source disposed within the housing, the UV radiation source including a UV lamp configured to provide UV radiation to the enclosure; directing a flow of air into a near field ambient environment of the UV lamp with a temperature control device; and maintaining a temperature of the UV lamp within an optimal temperature range.

### BRIEF DESCRIPTION OF THE DRAWINGS

The foregoing and other features and advantages will be apparent from the following description of the disclosure as illustrated in the accompanying drawings. The accompanying drawings, which are incorporated herein and form a part of the specification, further serve to explain the principles of the disclosure and to enable a person skilled in the pertinent art to make and use the disclosure. The drawings are not necessarily to scale.

FIG. 1A is perspective view of a sanitizing apparatus according to embodiments hereof

FIG. 1B is perspective view of a sanitizing apparatus according to embodiments hereof

FIG. 2 is a graph illustrating example UV lamp output at various lamp bulb surface temperatures.

FIG. 3A is a perspective view of a UV radiation source and associated temperature regulating components according to embodiments hereof.

FIG. 3B is a perspective view of a sanitizing apparatus according to embodiments hereof

FIG. 3C is a perspective view of a UV radiation source and associated temperature regulating components according to embodiments hereof.

FIG. 4A is a perspective view of a UV radiation source and associated temperature regulating components according to embodiments hereof.

FIG. 4B is a perspective view of a sanitizing apparatus according to embodiments hereof

FIG. 5A is a perspective view of a UV radiation source and associated temperature regulating components according to embodiments hereof.

FIG. 5B is a perspective view of a sanitizing apparatus according to embodiments hereof

FIG. 6A is a perspective view of a UV radiation source and associated temperature regulating components according to embodiments hereof.

FIG. 6B is a perspective view of a sanitizing apparatus according to embodiments hereof

FIGS. 7A, 7B, 7C and 7D illustrate a sanitizing apparatus configured to provide sanitized air to an environment according to embodiments hereof.

FIG. 8 is a flow chart of a method of sanitizing objects consistent with embodiments hereof.

### DETAILED DESCRIPTION

Specific embodiments of the present disclosure will now be described with reference to the Figures, wherein like reference numbers indicate identical or functionally similar elements. The following detailed description is merely exemplary in nature and is not intended to limit the disclosure or the application and uses of the disclosure. A person skilled in the relevant art will recognize that other configurations and arrangements can be used without departing from the scope of the disclosure. Although the description and drawings of the embodiments hereof exemplify a sanitation apparatus as applied to sanitizing wheelchairs, the disclosure may also be used in other sanitizing applications, for example to sanitize shopping carts as used in commercial retail establishments. Furthermore, there is no intention to be bound by any expressed or implied theory presented in the preceding technical field, background, brief summary or the following detailed description.

A sanitizing apparatus and system that provides a dual sanitizing functionality is described herein. In embodiments, the sanitizing apparatus may be used to sanitize an object (i.e., a wheelchair) or objects (i.e., a rack of instruments) by placing the object(s) within a sanitizing chamber configured with one or more UV radiation sources. In embodiments, the sanitizing apparatus may be used to sanitize the air around the apparatus, by directing a flow of sanitized air into the surrounding environment. With this dual functionality, the sanitizing apparatus is said to have both object and air sanitizing capability

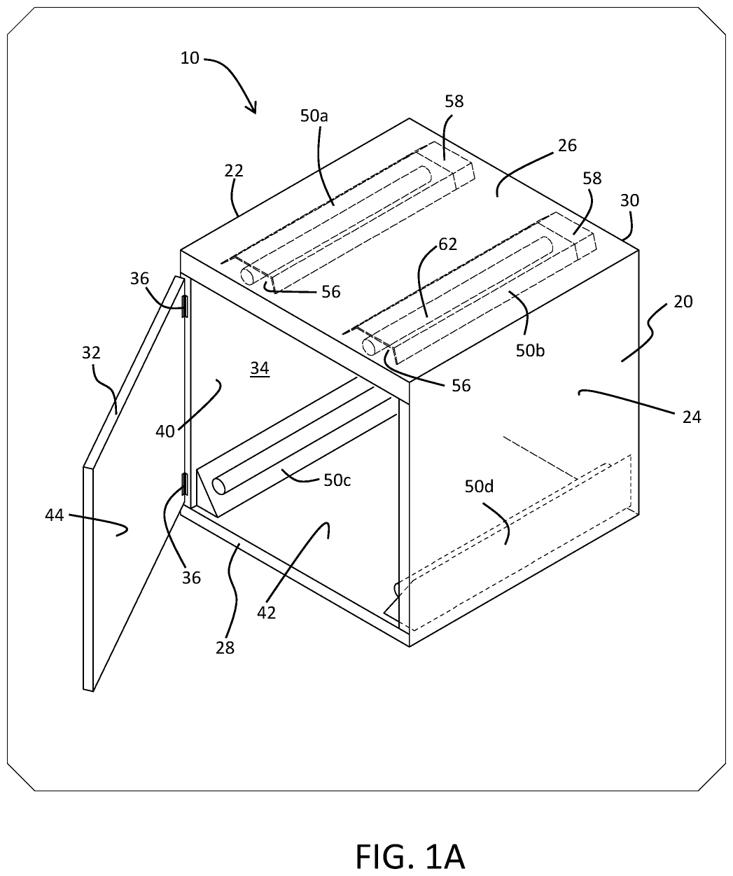

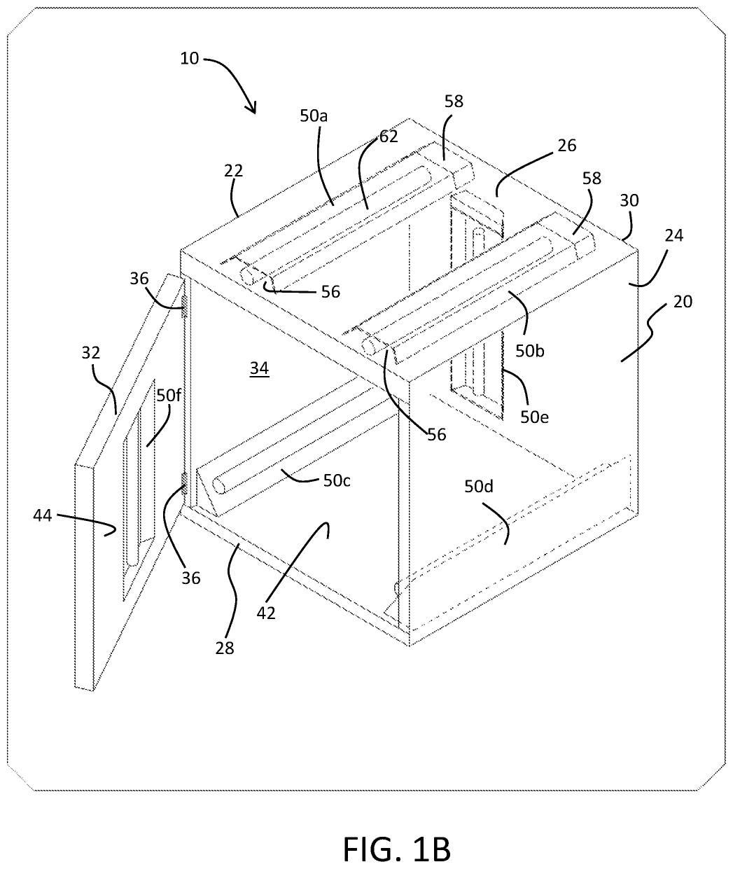

Turning now to FIG. 1A, shown is a sanitizing apparatus 10. The sanitizing apparatus 10 is generally configured as an enclosure defined by a housing 20. The housing 20 is arranged with a first and second side wall 22, 24, a top wall 26, a bottom wall 28, and a rear-wall 30. The first and second side walls 22, 24, the top wall 26, the bottom wall 28, and the rear-wall 30 are fixedly arranged into the enclosure as shown. The housing 20 also includes a forward wall 32. The forward wall 32 is hingedly connected to one of the first and second side walls 22, 24, so as to permit objects to be placed into the sanitizing environment 34 defined by the housing 20. As shown, the forward wall 32 is connected to the first side wall 22 using a pair of suitable hinges 36. Although standard barrel-type hinges are shown, it will be appreciated that a variety of hinge types may be suitably implemented. The forward wall 32 is generally configured to fully open in a manner that permits mobile objects to be placed into the sanitizing environment 34. To facilitate placement of mobile objects into the sanitizing environment 34, the sanitizing apparatus 10 may include a ramp or similar structure (not shown). In further embodiments, the forward wall 32 includes an opening and a door configured to selectively cover the opening.

The construction of the housing 20 is not intended to be limited to one specific construction methodology. The housing 20 may be of various configurations, using a variety of construction materials to achieve the desired function. For instance, in one exemplary form, the housing 20 may be an open framed structure using metal (i.e., aluminum) T-slot tubing, with the first and second side walls 22, 24, the top wall 26, and the rear and forward walls 30, 32 being covered using a suitable material that is opaque and/or generally non-transmissive to UV radiation. For instance, the framed structure may be covered using aluminum plate. While the bottom wall 28 may be covered with a similar opaque and/or generally non-transmissive material as used on the various walls, a more robust material may be selected to support and/or provide the required durability with respect to the objects that may be placed within the sanitizing environment 34. It will also be appreciated that the shape of the sanitizing apparatus 10 may take on a variety of forms. Although shown as having six planar sides arranged as an elongated (rectangular) box, other shapes may be implemented. For example, some embodiments of the sanitizing apparatus may have at least some of the walls formed with curved or otherwise non-planar surfaces, in particular where such a configuration is shown to enhance operation/effectiveness of the sanitizing process.

The housing 20 may additionally include a set of internal reflective elements having UV reflective properties. The set of internal reflective elements may be disposed on an inside surface of one or more of the first and second side walls 22, 24, the top wall 26, the bottom wall 28, and the rear and forward walls 30, 32 as deemed necessary to effectively distribute the UV radiation within the sanitizing environment 34. Each element of the set of internal reflective elements may be a panel separately applied to an inside surface of a respective wall section, or each element may be an applied reflective paint, film, or other surface coating. In the exemplary embodiment shown in FIG. 1A, the housing 20 includes a full compliment of internal reflective elements on at least a portion of all inside surfaces. In the view shown, a first-side wall internal reflective element 40 is disposed on an inside surface of the first side wall 22, a bottom internal reflective element 42 is disposed on an inside surface of the bottom wall 28, and a forward internal reflective element 44 is disposed on an inside surface of the forward wall 32. A variety of materials may be used for the internal reflective element. The desired characteristic of the internal reflective element is to be non-absorptive, dispersive, and reflective of the impinging UV radiation, with minimal reduction in radiation intensity. In a non-limiting example, the internal reflective elements may be formed of expanded Polytetrafluoroethylene (e-PTFE) or sintered PTFE.

The sanitizing apparatus 10 includes at least one UV radiation source. In the exemplary embodiment shown in FIG. 1A, the sanitizing apparatus 10 includes four UV radiation sources 50a-50d. First and second UV radiation sources 50a, 50b are mounted upon an inside surface of the top wall 26; a third UV radiation source 50c is mounted along at least a portion of the interface between the first side wall 22 and the bottom wall 28; a fourth UV radiation source 50d is mounted along at least a portion of the interface between the second side wall 24 and the bottom wall 28. It will be appreciated that the arrangement and manner of mounting the UV radiation sources may take on a variety of configurations. In some embodiments, a fewer number or a greater number of UV radiation sources 50 may be included. The location of the UV radiation sources may also vary from that exemplified, depending on the number and type of UV radiation source being used. For instance, an alternative arrangement is shown in FIG. 1B, where the sanitizing apparatus 10 is configured similarly to the embodiment of FIG. 1A, with the addition of a fifth UV radiation sources 50e mounted on the rear wall 30, and a sixth UV radiation source 50f mounted on the forward wall 32. Returning now to FIG. 1A, the UV radiation source 50 includes a UV lamp 62. Exemplary UV lamps include, but are not limited to low-pressure mercury lamps, high-pressure mercury lamps, excimer lamps, and light-emitting diode (LED) lamps. Depending on the lamp type selected, each UV radiation source 50 may include one or more UV lamps 62 to provide the desired dose of UV radiation.

The type of UV lamp is also selected based on its ability to provide a germicidal effect. Germicidal effects may be achieved through the use of ultraviolet-C (UV-C) radiation in the range of 200 nm to 280 nm. The lamp(s) selected for each of the UV radiation sources 50 may be UV-C lamp(s) having a radiation output in the range of 200 nm to 280 nm. In further embodiments, UV-C lamp(s) consistent with the embodiments herein may provide radiation at wavelengths between approximately 240 nm and 270 nm or may provide radiation at about 254 nm. In further embodiments, UV lamps that may be employed are not limited to UV-C lamps. UV lamps providing a germicidal effect through use of UV radiation outside of UV-C radiation (i.e., higher than 280 nm or lower than 200 nm) may also be selected. Accordingly, any UV lamps consistent with the provision of germicidal UV radiation may be selected according to embodiments herein.

In embodiments, the UV radiation sources 50 are configured to provide sterilizing or germicidal UV radiation to the enclosure defined by the housing. As discussed above, UV lamps consistent with embodiments herein may be provided in a range of germicidal radiation wavelengths. UV lamps 62 consistent with this disclosure may be “cycle-start” lamps designed for intermittent use. In further embodiments, UV lamps 62 consistent with this disclosure may be continuous use UV lamps designed for continuous use.

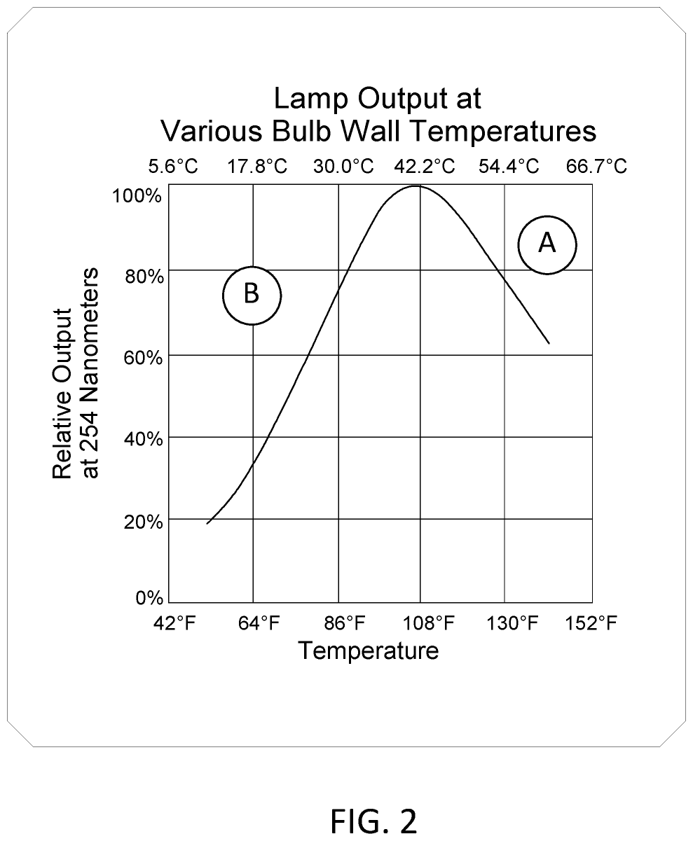

Each of the UV radiation sources 50 emit UV radiation into the housing 20, thereby irradiating any object(s) contained within the sanitizing environment 34. A near-field ambient environment 56 occupies the area immediately surrounding the UV lamps 62 of each UV radiation source 50. For certain UV lamps, for example, low-pressure mercury lamps, UV lamp output is a function of the lamp bulb surface temperature, where optimal lamp output is achieved at a lamp bulb surface temperature of about 40° C. This relationship between bulb surface temperature to UV lamp output for low-pressure mercury lamps is shown in FIG. 2. During operation, UV lamps heat up. A UV lamp that cycles on at room temperature (e.g., 25° C.) gradually increases in output until it reaches a maximum output and then begins to decrease in output as it continues to heat up. An optimal temperature range may be defined as a temperature range within which a UV lamp output is at least 80%, 90%, 95%, or 98% of a maximum temperature dependent output. Accordingly, increased or optimized lamp output may be achieved by maintaining the temperature of the near-field ambient environment 56, and thus the bulb surface temperature of each of the UV lamps 62 provided in the UV radiation sources 50, in the optimal range. As described below, one or more temperature control devices may be included in the sterilizing apparatus to maintain the temperature within the optimal temperature range.

With reference again to FIG. 1A, the first and second UV radiation sources 50a, 50b include a near-field ambient environment temperature control device 58. Although not visible in FIG. 1A, similar temperature control devices are included for the third and fourth UV radiation sources 50c, 50d. The temperature control devices 58 establish temperature control of the near-field ambient environment 56 of each of the UV radiation sources 50. In general terms, the temperature control devices 58 are configured to direct into the near field ambient environment 56 a source of air. It will be appreciated that the temperature control device 58 may take on a variety of forms. For instance, and as will be described in greater detail below, the temperature control devices 58 may be, for example, a fan or blower, and may include one or more cooling and/or heating elements to introduce additional cooling/heating as necessary.

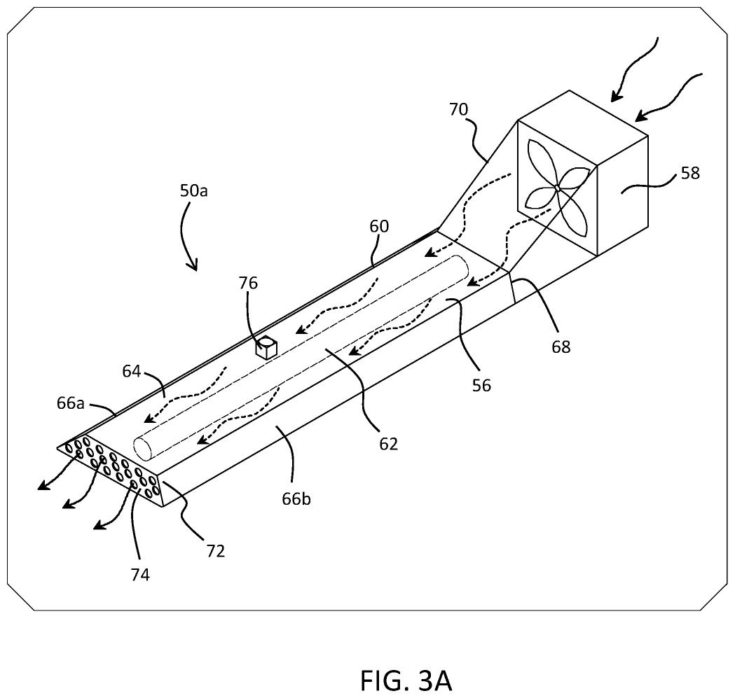

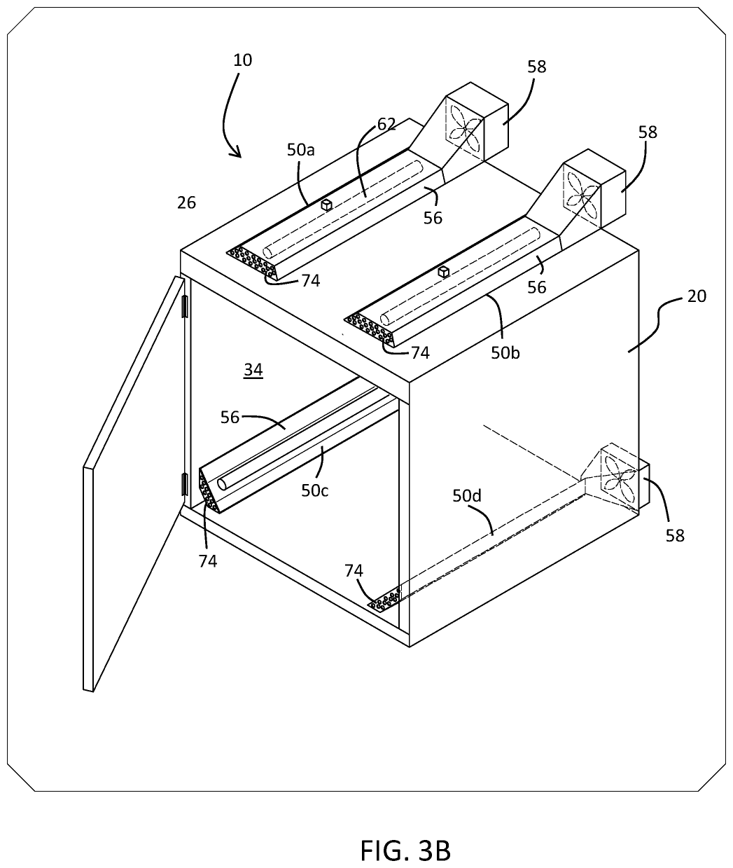

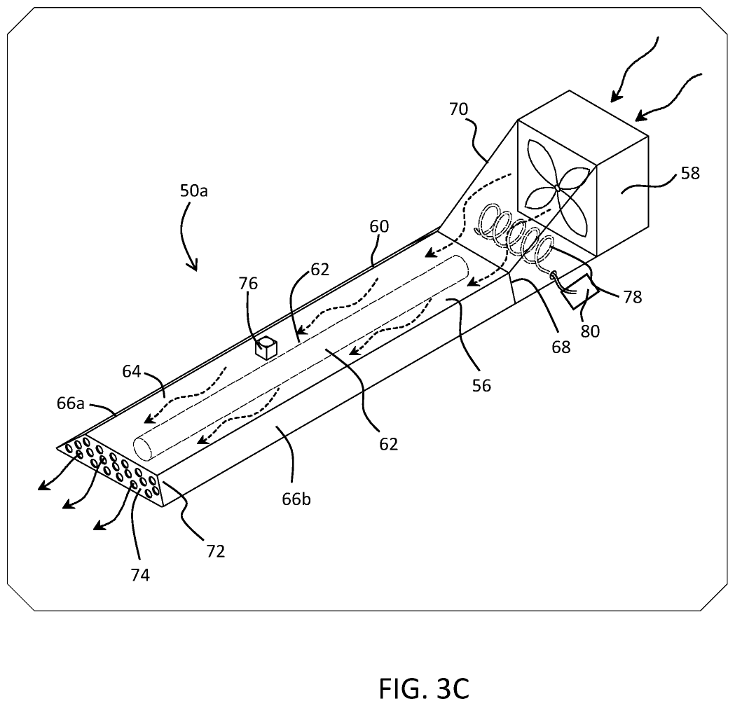

FIGS. 3A and 3B illustrate an exemplary embodiment of the UV radiation source 50 and associated temperature regulating arrangement. FIG. 3A illustrates the UV radiation source 50a and the temperature control device 58 in isolation. FIG. 3B illustrates the UV radiation source 50a and the temperature control device 58 operably mounted upon the housing 20. The housing 20 is constructed in much the same way as that previously described with regard to the embodiment of FIG. 1A. Accordingly, only differences in the construction of the housing 20 will be specifically noted. As the four UV radiation sources 50a-50d are configured largely in the same way, reference is made to UV radiation source 50a for explanation, but it will be appreciated that the construction detailed herein is equally applicable to the other UV radiation sources 50b-50d. The UV radiation source 50a has an elongate structure and includes a reflector unit 60 in which one or more UV lamps 62 are mounted. As shown, a singular elongate UV lamp 62 is shown, an example of which may be a low-pressure mercury lamp. The reflector unit 60 is a 3-paneled structure, having a longitudinal back panel 64, and two opposing longitudinal side-panels 66a, 66b. The one or more UV lamps 62 are mounted centrally relative to the opposing longitudinal side-panels 66a, 66b. The reflector unit 60 is open on the side opposite the longitudinal back panel 64. In some embodiments, a UV-transmissive protective cover may be used to cover at least a portion of this opening. The reflector unit 60 has a first end 68 that is operably associated with the temperature control device 58 through a suitable conduit 70. The first end 68 serves as the inlet for the source of air to be introduced into the near field ambient environment 56 of the UV radiation source 50 by the temperature control device 58. The temperature control device 58 is a fan, blower, or other device suitable for generating a flow of air. In embodiments, as illustrated in FIG. 3B, the temperature control device 58 is a box-type fan and is generally positioned to pull air from the surrounding ambient environment external to the housing 20 and is configured to direct the air through the near field ambient environment 56. In this way, as the ambient air external to the housing 20 is generally cooler than the optimal lamp bulb surface temperature, the stream of air being delivered to the near field ambient environment 56 cools the lamp, preventing it from rising above the optimal temperature range. Opposite the first end 68, the reflector unit 60 has a second end 72 that serves as the outlet for the air flowing through the near field ambient environment 56. The second end 72 may include a diffuser 74.

As shown in FIG. 3B, the first and second UV radiation sources 50a, 50b are externally mounted upon the top wall 26, while the third and fourth UV radiation sources 50c, 50d are internally mounted, similar to that described relative to the embodiment of FIG. 1A. It will be appreciated that where UV radiation sources 50 are externally mounted upon the housing 20, the wall structure upon which the UV radiation source 50 is located will provide a suitably sized aperture to permit the passage of UV radiation from the UV radiation source 50 into the sanitizing environment 34. Continuing with FIG. 3B, each of the UV radiation sources 50 are configured with a dedicated temperature control device 58. It will be appreciated, however, that other configurations of supplying air from a box-type fan may be implemented, for instance where a plenum or manifold is used to supply air to a plurality of UV radiation sources. In embodiments, any or all of the UV radiation sources 50 further include diffusers 74 disposed at a second end of each of the UV radiation sources 50. With respect to the first and second UV radiation sources 50a, 50b, the UV radiation sources and therein the associated diffusers 74 are external to the top wall 26 of the housing 20. Accordingly, the stream of air directed through the near field ambient environment 56 of each of the first and second UV radiation sources 50a, 50b vents to the external environment, relative to the housing 20. With respect to the third and fourth UV radiation sources 50c, 50d, the UV radiation sources and therein the associated diffusers 74 are internal to the housing 20. Accordingly, the stream of air directed through the near field ambient environment 56 of each of the third and fourth UV radiation sources 50c, 50d vents to the internal environment, relative to the housing 20. As the stream of air flowing through the near field ambient environment 56 of each UV radiation source 50 is also sanitized by the respective UV lamp(s) contained therein, the exhaust air is a source of beneficial sanitized air. In the case of the first and second UV radiation sources 50a, 50b, the sanitized air is released to the ambient environment external to the housing 20, therein benefiting the area around the sanitizing apparatus 10. In the case of the third and fourth UV radiation sources 50c, 50d, the sanitized air is released within the housing 20. As the air was drawn from the external environment by the temperature control device 58 and released into the area defined by the housing 20, not only is sanitized air being used to envelop the object being sanitized, the release of sanitized air within the housing 20 establishes a positive pressure environment within the housing 20, therein reducing the likelihood of contaminants being drawing back into the housing 20 while an object is being sanitized. Although not shown, the housing 20 may additionally include one or more vents to release sanitized air from the sanitizing environment 34. The vents may be adjustable, to enable control of the amount of positive pressure established within the housing 20. The number and arrangement of the radiation sources 50a-50d including diffusers 74 that direct air to the environment surrounding the housing 20 or the environment within the housing 20 is not confined to that discussed with respect to FIG. 3B. Each of the radiation sources 50a-50d may be configured with a diffuser to direct air to either the environment surrounding the housing 20 or to the environment within the housing 20

The temperature control device 58 as shown in FIGS. 3A and 3B is configured to direct a flow of air through the near field ambient environment of the UV lamps 62 of each respective UV radiation source 50. As previously mentioned, as the ambient air external to the housing 20 is generally cooler than the optimal lamp bulb surface temperature, the stream of air being delivered to the near field ambient environment 56 serves to cool the UV lamp(s), to prevent them from rising above the optimal temperature range. In this particular configuration, the temperature control device 58 is suited to provide a cooling function, therein serving to correct the lamp bulb surface temperature when it exits the optimal temperature range and rises into region A of the temperature profile shown in FIG. 2. In embodiments, the temperature control device 58 further includes an additional heating and/or cooling element, to provide additional heating/cooling functionality. With reference to FIG. 3C, shown is a temperature control device 58 configured similarly to the temperature control device shown in FIG. 3A, with the exception that it includes an additional temperature control element 78. Although a single temperature control element 78 is shown, it will be appreciated that one or more such temperature control elements 78 may be incorporated into the temperature control device 58. The temperature control element 78 may include a heating element, a cooling element, or both, depending on the type of temperature control required. For instance, the temperature control device 58 may incorporate one or more cooling elements to further address temperature adjustments necessary when the lamp bulb surface temperature rises into region A of the temperature profile (FIG. 2). Similarly, the temperature control device 58 may incorporate one or more heating elements to provide a heating function, therein serving to reduce the time required to achieve the optimal lamp bulb surface temperature, for example as would be the case upon lamp start-up, when the lamp bulb surface temperature is generally within region B of the temperature profile (FIG. 2). It will be appreciated that the temperature control device may incorporate a combination of one or more heating and cooling elements, as deemed necessary for the particular application.

The operation of the temperature control device 58 may be controlled through a temperature control circuit 80, based on observed conditions relative to a preferred operational state. The temperature control circuit 80 may include a microprocessor including software, an FPGA including firmware, and/or a hardware-based circuit. For instance, the rate of the air flow delivered by the temperature control device 58 may be controlled based on temperature readings in the near field ambient environment 56 as detected by one or more temperature control sensors 76. Where the temperature reading at the temperature control sensor 76 falls outside of the optimal temperature range within region A of the temperature profile (FIG. 2), the rate of air flow through the near field ambient environment 56 may be increased until the temperature within the near field ambient environment 56 falls below an upper threshold of a predetermined operational temperature range. This cooling effect may be further enhanced where the temperature control device 58 additionally includes one or more cooling elements, in which case the temperature control circuit 80 would control the activation of these elements as required. Conversely, where the temperature reading at the temperature control sensor 76 falls out of the optimal temperature range within region B of the temperature profile (FIG. 2), the rate of air flow through the near field ambient environment 56 may be decreased (or stopped) until the temperature within the near field ambient environment 56 rises above a lower threshold of the optimal temperature range. This heating effect may be further enhanced where the temperature control device 58 additionally includes one or more heating elements, in which case the electronic circuitry would control the activation of these elements as required.

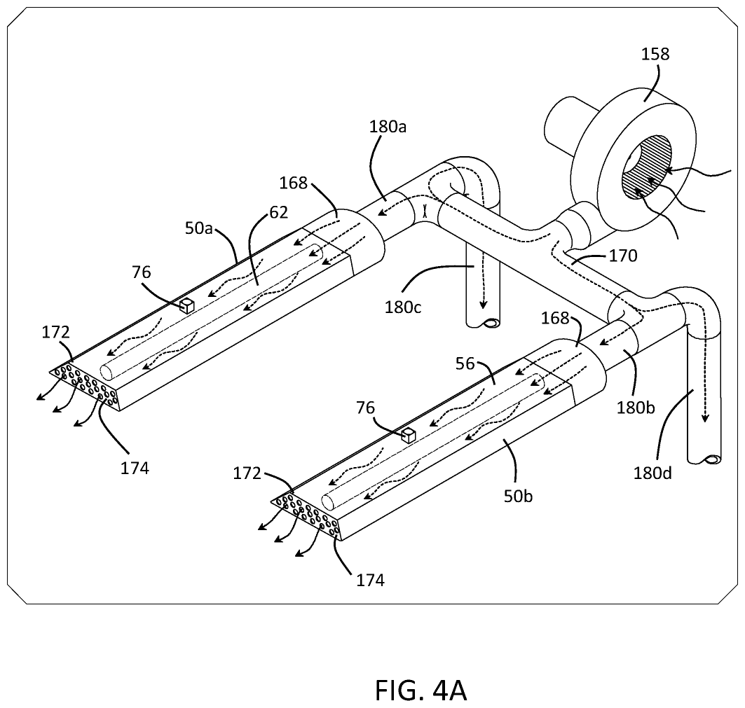

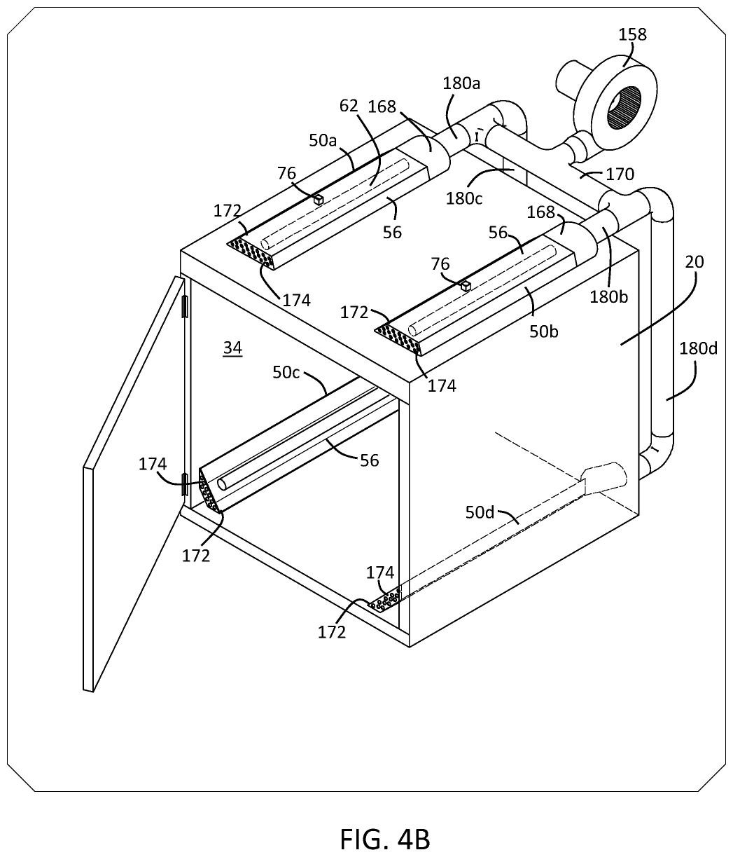

FIGS. 4A and 4B illustrate an alternative arrangement for the supply of air to the UV radiation sources 50. The arrangement is shown in isolation (FIG. 4A), and operably mounted (FIG. 4B) upon the housing 20. The housing 20 is constructed in much the same way as that previously described with respect to the embodiment of FIG. 1A. Accordingly, only differences in the construction of the housing 20 will be specifically noted. The UV radiation sources 50 are constructed and positioned relative to the housing 20 in a similar way as that previously described with respect to the embodiment of FIGS. 3A and 3B. The additions and/or differences described with respect to FIGS. 4A and 4B may be incorporated or combined with the features of any previous embodiment. A first end 168 of each of the UV radiation sources 50a, 50b are operably associated with the temperature control device 158 through a suitable conduit 170. The conduit 170 serves as a manifold to distribute the stream of air from a single temperature control device 158 into the near field ambient environment 56 of the UV lamps 62 of each of the UV radiation sources 50 connected thereto. As shown, the conduit 170 includes a first conduit section 180a to deliver air to the first UV radiation source 50a, a second conduit section 180b to deliver air to the second UV radiation source 50b, a third conduit section 180c to deliver air to the third UV radiation source 50c, and a fourth conduit section 180d to deliver air to the fourth UV radiation source 50d. The temperature control device 158 shown for providing the stream of air is, for example, a centrifugal-type fan, and is generally positioned to pull air from the surrounding ambient environment external to the housing 20 and is configured to direct the air through the near field ambient environment 56. Other aspects of the UV radiation sources 50a, 50b are similar to those previously described, including a diffuser 174 disposed on a second end 172 of each UV radiation source 50, and one or more temperature control sensors 76. The temperature control device 158 and/or operably associated air delivery conduits 170, 180 may additionally include one or more cooling elements, heating elements, and/or temperature control circuits (not shown) to further modulate the temperature of air being delivered to the near field ambient environment 56 of each of the UV radiation sources 50 connected thereto.

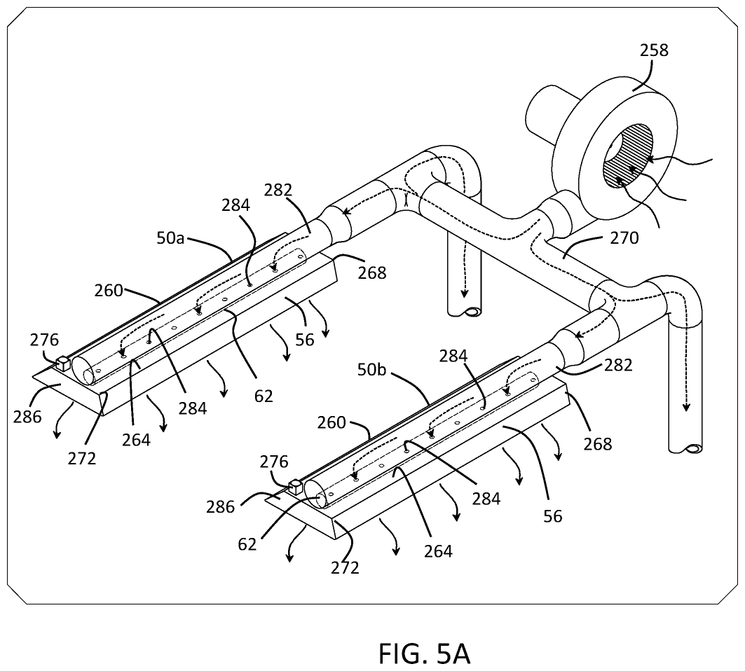

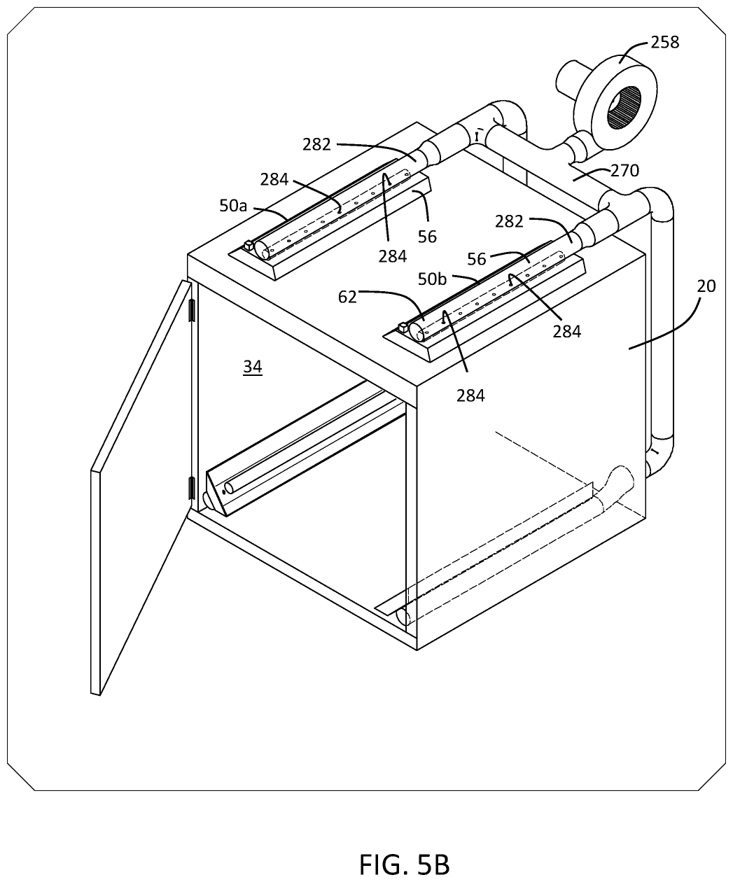

FIGS. 5A and 5B illustrate a further alternative arrangement for the supply of air to the UV radiation sources 50. The arrangement is shown in isolation (FIG. 5A), and operably mounted (FIG. 5B) upon the housing 20. The housing 20 is constructed in much the same way as that previously described with respect to the embodiment of FIG. 1A. Accordingly, only differences in the construction of the housing 20 will be specifically noted. The UV radiation sources 50 are constructed and positioned relative to the housing 20 in similar fashion as that previously described with respect to the embodiment of FIGS. 3A and 3B. Similarly, a conduit 270 is positioned relative to the housing 20 similarly to that previously described with respect to the conduit 170 shown in the embodiment of FIGS. 4A and 4B. The additions and/or differences described with respect to FIGS. 5A and 5B may be incorporated or combined with the features of any previous embodiment. Conduit 270 directs the stream of air through an air distribution pipe 282 operably connected to the longitudinal back panel 264 of each of the UV radiation sources 50. The air distribution pipe 282 of each of the UV radiation sources 50 fluidly communicates with the respective near field ambient environment 56 through a plurality of air holes 284 arranged therebetween. In this way, the stream of air being delivered from the temperature control device 258 envelops the UV lamp 62 from the series of air holes 284 distributed along the length of the reflector unit 260, instead of from one end thereof. To additionally promote the movement of air in the direction towards the UV lamps 62, the first and second ends 268, 272 of the reflector unit 260 include respective rear and forward closure panels 286 (rear closure panel on first end not visible). The temperature control device 258 shown for providing the stream of air may be a centrifugal-type fan as illustrated in FIG. 5A, and/or may be any other suitable type of fan, blower, or air flow generation device, and is generally positioned to pull air from the surrounding ambient environment external to the housing 20 and is configured to direct the air through the near field ambient environment 56 of the UV lamps 62. All other aspects are as previously described, including the provision of one or more temperature control sensors 276. The temperature control device 258 and/or operably associated air delivery conduits 270, 282 or may additionally include one or more cooling elements, heating elements, and/or temperature control circuits (not shown) to further modulate the temperature of air being delivered to the near field ambient environment 56 of each of the UV radiation sources 50 connected thereto.

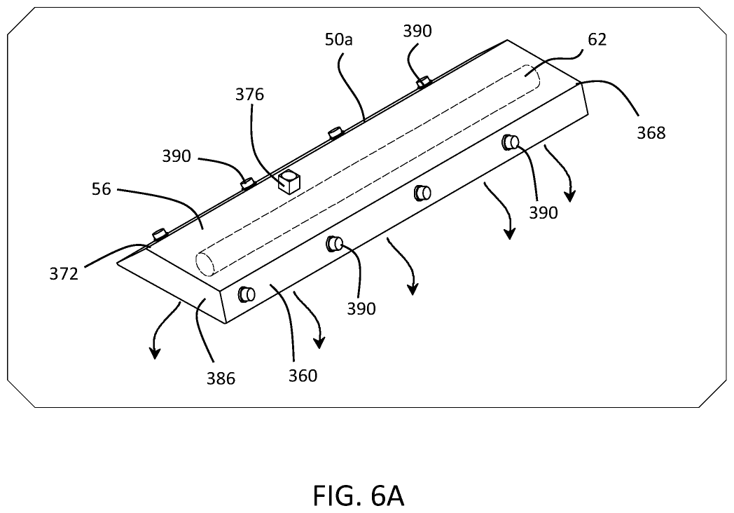

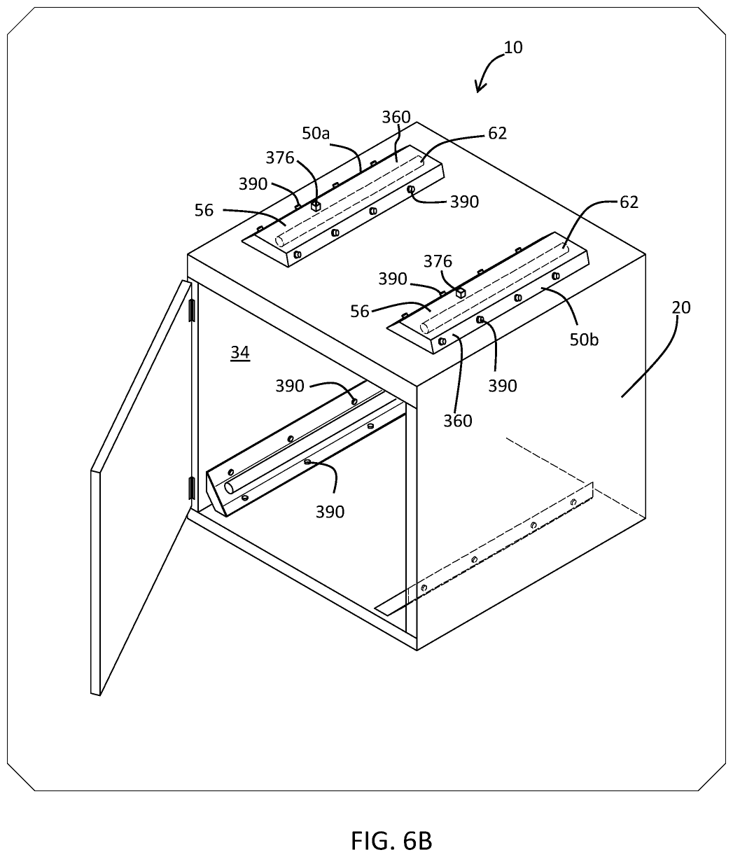

FIGS. 6A and 6B illustrate a further alternative arrangement for the supply of air to the UV radiation sources 50. The arrangement is shown in isolation (FIG. 6A), and operably mounted (FIG. 6B) upon the housing 20. The housing 20 is constructed in much the same way as that previously described with respect to the embodiment of FIG. 1A. Accordingly, only differences in the construction of the housing 20 will be specifically noted. The UV radiation sources 50 are constructed and positioned relative to the housing 20 in a similar fashion as that previously described with respect to the embodiment of FIGS. 3A and 3B. The additions and/or differences described with respect to FIGS. 6A and 6B may be incorporated or combined with the features of any previous embodiment. The air for each of the UV radiation sources 50 is supplied locally through the use of one or more electronic temperature control units 390 mounted on each respective reflector unit 360. The temperature control units 390 are selected on their ability to provide a heating and/or cooling effect through radiant/convective means to the near field ambient environment 56 of the UV lamp 62 of each respective UV radiation source 50. Exemplary temperature control units 390 configured to provide a heating effect include but are not limited to electric heating coils. Exemplary temperature control units 390 configured to provide a cooling effect include but are not limited to thermoelectric cooler modules that provide cooling based on the Peltier effect. It will be appreciated that a combination of heating and cooling temperature control units 390 may be arranged to provide the required temperature regulation for each of the UV radiation sources 50. Since the heating and/or cooling is provided through radiant/convective means, the air supply system (i.e., the conduit) does not form part of the construction. Similar to the UV radiation sources 50 described above with respect to the embodiment of FIGS. 5A and 5B, to promote the movement of temperature regulated air in the direction towards the UV lamps 62, the first and second ends 368, 372 of the reflector unit 360 include respective rear and forward closure panels 386 (rear closure panel on first end not visible). All other aspects are as previously described, including the provision of one or more temperature control sensors 376. Additionally, a temperature control circuit as described above (not shown) may be employed to control the temperature control units 390. Further, the temperature control units 390 may be employed with any of the previously described temperature control devices 58, 158, 258 etc. and/or any other features previously described with respect to the foregoing embodiments.

In embodiments, the UV radiation sources described herein may use a type of bulb that requires continuous operation, increasing the likelihood that the UV radiation source may be energized when the sanitizing environment is not being used to sanitize an object. For example, in some embodiments a low-pressure mercury amalgam lamp may be used to obtain a broader optimal temperature range. Such lamps are generally intended for continuous operation and may fail more quickly under cycle-start operation conditions.

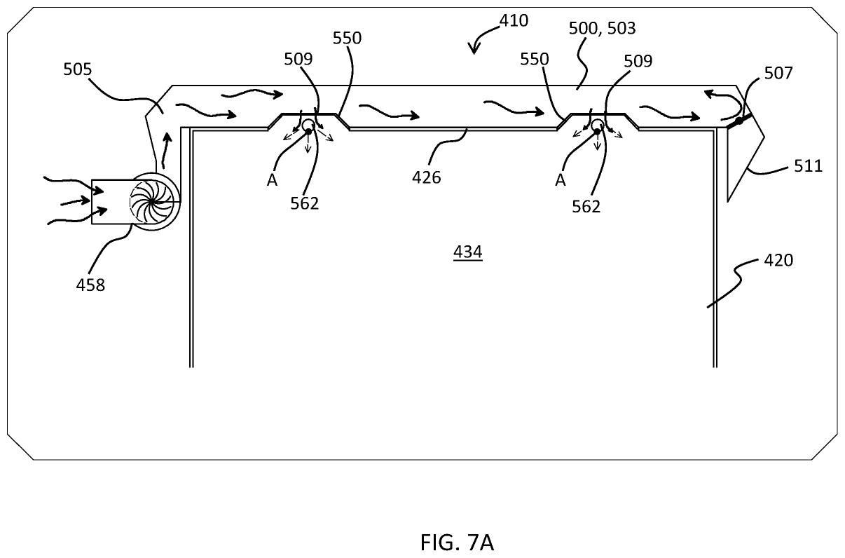

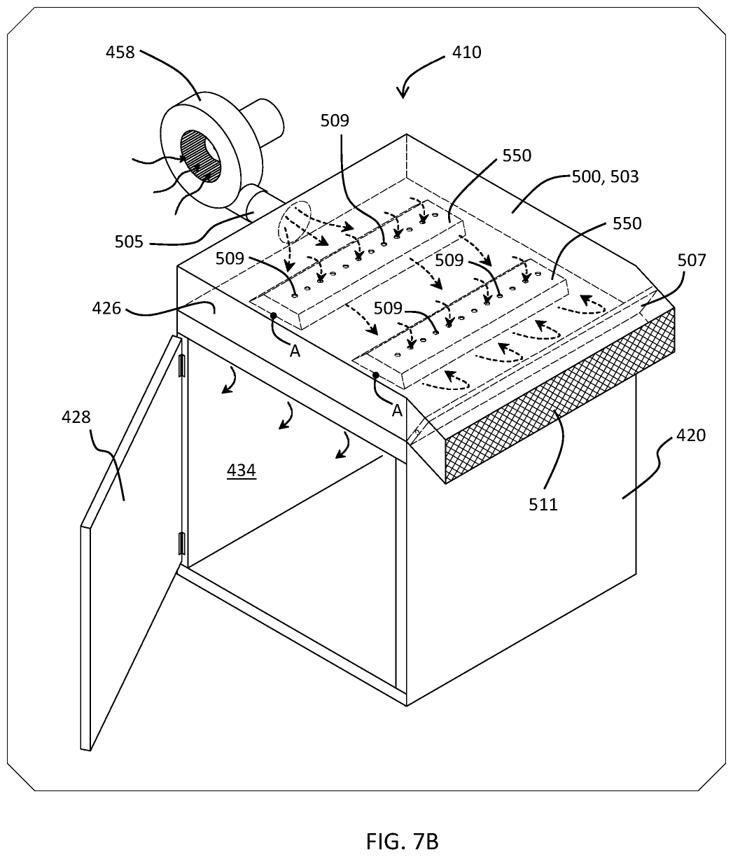

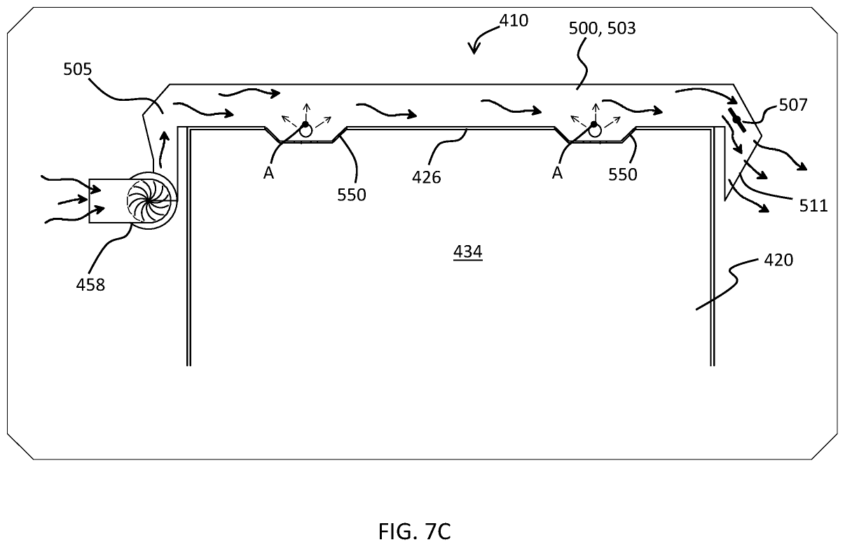

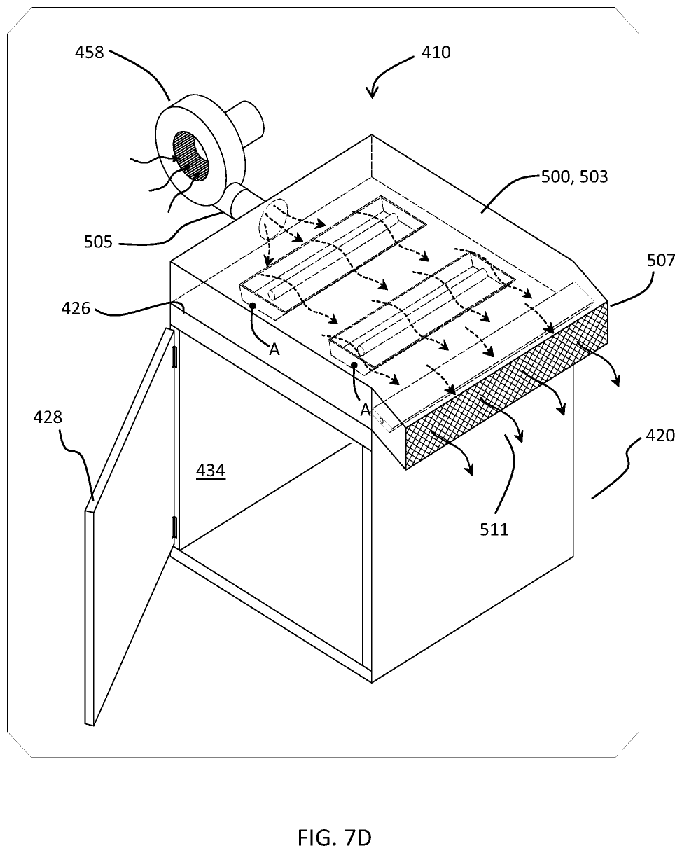

FIGS. 7A, 7B, 7C and 7D illustrate a sanitizing apparatus 410 configured for dual functionality. More specifically, in a first mode, an object irradiation mode, the sanitizing apparatus 410 is configured to irradiate an object or objects placed within the housing 420, i.e., within the sanitizing environment 434 contained therein. In a second mode, an air irradiation mode, the sanitizing apparatus 410 is configured to irradiate a volume of air within a plenum, the sanitized air thereafter being released from the sanitizing apparatus 410 into the surrounding area, therein providing means to cleanse the air, for example in a room designated for sanitizing objects. The sanitizing apparatus 410 is constructed in a manner that permits a user to easily move from one mode to the other as deemed necessary.

As shown, the housing 420 is constructed in much the same way as that previously described with respect to the embodiment of FIG. 1A. Accordingly, only differences in the construction of the housing 420 will be specifically noted. Mounted on the top wall 426 is a secondary enclosure 500 that defines a plenum 503, configured to receive a volume of air from a temperature control device 458, through a plenum inlet 505. As shown, the temperature control device 458 may be a centrifugal-type fan and is generally positioned and configured to pull air from the surrounding ambient environment external to the housing 420. Other types of fans, blowers, and/or air flow generation devices may be suitably implemented, for example a box-type fan as shown in the embodiment of FIGS. 3A and 3B. The temperature control device 458 and/or operably associated air delivery plenum 503 may include one or more cooling elements, heating elements, and/or temperature circuits (not shown) to further modulate the temperature of air flowing therethrough. The plenum 503 is arranged to direct the volume of air into and/or across the UV lamps 62 one or more rotatable UV radiation sources 550 arranged on the top wall 426. The one or more rotatable UV radiation sources 550 are rotatable about a longitudinal axis A, to irradiate either the sanitizing environment 434, or the plenum 503, depending on the selected mode of operation. In the object irradiation mode, as depicted in FIG. 7A, the UV radiation sources 550 and associated UV lamps 562 are configured such that the produced UV radiation is provided to the sanitizing environment 434. The plenum 503 additionally includes a gate 507 that may be positioned in either a closed position, as shown in FIGS. 7A and 7B, or an open position as shown in FIGS. 7C and 7D. In the closed position, the gate 507 serves to direct the flow of air into the sanitizing environment 434 of the housing 420. As shown, the UV radiation sources 550a, 550b each include a plurality of air holes 509 that fluidly connect the plenum 503 to the sanitizing environment 434. With the gate 507 in the closed position, the positive pressure that builds within the plenum 503 serves to drive the flow of air through the air holes 509, and into the near field ambient environment of the UV lamps 562 of each respective UV radiation source 550. This configuration represents the object irradiation mode of operation, in which the UV radiation sources 550 are rotated into the downward irradiating orientation, as shown in FIG. 7A. With the gate 507 in the open position, the plenum 503 primarily serves as a flow through chamber that directs the stream of air across the UV radiation sources 550, in the direction towards and out through the plenum outlet 511. This configuration represents the air irradiation mode of operation, in which the UV radiation sources 550 are rotated into the upward irradiating orientation, as shown in FIG. 7C. As discussed above, in the object irradiation mode, with the UV radiation source 550 rotated into the downward irradiating orientation, objects placed within the sanitizing environment 434 are irradiated and therein sanitized. In the air irradiation mode, with the UV radiation source 550 rotated into the upward irradiating orientation, the air passing through the plenum 503 is subject to UV dosing, after which it is released through the plenum outlet 511, therein providing the ambient environment surrounding the sanitizing apparatus 410 with a beneficial source of cleansed air.

The shift between the first and second modes may be facilitated through the use of manual actuators operable by the user, to rotate the UV radiation sources 550 and the gate 507 into the required position. For example, the UV radiation sources 550 and the gate 507 may each be configured with a rotatable axel, access to which may be gained via a handle disposed on an exterior of the housing 20. The rotation of the UV radiation sources 550 and the gate 507 may be operably connected, for example through a mechanical linkage, so as to ensure the correct positioning relative to these adjustable elements. The shift between the object irradiation and air irradiation modes may also be facilitated through the use of electronic, hydraulic or pneumatic actuators that are operably connected with an electronic control circuit, having a user interface that permits for the selection of the desired mode of operation. Regardless of the mode, the air flowing past the UV lamp 562 of the UV radiation source 550 is sanitized. The UV radiation source 550 and the gate 507 are configured to selectively direct a flow of the sanitized air either into the enclosure (in an object irradiation mode) or into a local environment of the sanitizing apparatus (in an air irradiation mode). In further embodiments, the rotation of the UV radiation sources 550 and the gate 507 may be operably governed by a positioning of the front wall 428 or a door. The UV radiation source 550 and the gate 507 may be configured to rotate, through mechanical linkages, pneumatic actuators, hydraulic actuators, electronic actuators, etc., into the object irradiation mode position when the front wall 428 or door is closed and into the air irradiation position when the front wall 428 or door is open. Such rotation may protect operators from exposure to UV radiation when the door is opened.

In embodiments, the sanitizing apparatus 410 may be configured to supply sanitized air in an air sanitizing mode to an environment local to the sanitizing apparatus 410. The sanitizing apparatus 410 may provide sanitized air through the plenum 503 to the area surrounding the sanitizing apparatus 410. In further embodiments, the sanitizing apparatus 410 may be configured to supply sanitized air in an air sanitizing mode to remote environments, via the use of duct work or integration into a local HVAC system.

FIG. 8 is a flow chart of a method 800 of sanitizing objects with a sanitizing apparatus consistent with embodiments hereof. The method 800 may be carried out with any sanitizing apparatus described herein with any suitable combination of features and/or with any other sanitizing apparatus providing the functional capacity described herein.

In an operation 802, an object or objects are placed in an enclosure of a sterilizing apparatus. The object or objects are placed in the enclosure to be sterilized and the door or front wall is closed.

In an operation 804, a UV radiation source, or multiple UV radiation sources, disposed within the housing are activated to provide UV radiation to the enclosure via UV lamps. The UV lamps are selected to provide UV radiation suitable for sanitizing or sterilizing the objects placed in the enclosure.

In an operation 806, a temperature control device is employed to direct a flow of air into the near field ambient environment of a UV lamp associated with the UV radiation source. In embodiments that include multiple UV radiation source, multiple temperature control devices may be used, or a single temperature control device may be used with ducting or conduits to split and redirect the flow of air to each of the UV radiation source.

In an operation 808, a temperature of the UV lamp or lamps are maintained within an optimal temperature range. As discussed above, the optimal temperature range is the temperature range within which the UV lamp is able to produce at least 80%, 90%, 95%, or 98% of its maximum temperature dependent output. As discussed above, the temperature control device may employ one or more temperature sensors, one or mote heating and/or cooling elements, and a temperature control circuit to assist in maintenance of the optimal temperature range.

The various embodiments of the sanitizing apparatus detailed above are exemplified as scaled to receive larger objects, such as wheelchairs, shopping carts, and other similar items. The disclosed technology may also be scaled for larger as well as smaller applications. For instance, the sanitizing apparatus could be scaled to receive elongate objects such as hospital beds and ambulance gurneys. A sanitizing apparatus having a larger sanitizing environment may also be preferable where multiple objects are to be sanitized at the same time. Smaller applications may find application in residential environments, or medical offices. For instance, the sanitizing apparatus may be sized similarly to a microwave, to permit for sanitizing smaller objects such as utensils, phones, and other objects commonly touched by multiple people. In some embodiments, the sanitizing apparatus may be configured to receive one or more rack- or cart-type structures to facilitate the placement of multiple smaller objects in a larger sanitizing apparatus. The rack-type structures may be constructed in a self-supporting configuration and may include castors to facilitate movement in/out of the sanitizing environment. The rack-type structures may also be configured to cooperate with tracks or similar hardware located on the interior surfaces of the sanitizing apparatus.

The various embodiments have demonstrated the stream of air as being generated by various types of fans, as well as a thermoelectric source. The various embodiments have also considered the use of additional heating and/or cooling elements to further modulate the temperature of the air. As previously stated, the air serves to maintain the UV radiation sources within a preferred optimal temperature range. It will be appreciated that the aforementioned sources of air are not meant to be limiting, as the stream of air may be supplied in a variety of ways. For instance, some embodiments of the sanitizing apparatus, both the singular object sanitizing form, as well as the dual functionality form may make use of air derived from a central HVAC system, or a high-capacity compressed air source.

Although only the temperature control device shown in FIG. 3C exemplifies the use of an additional temperature control element, each of the embodiments shown herein (FIGS. 4A through 7D) may also incorporate additional heating and/or cooling elements and temperature control circuitry into the temperature control device, to achieve the desired temperature control. The addition of temperature control elements may be dictated by the particular implementation of the sanitizing apparatus, the type of UV lamp being used, and the environment in which the sanitizing apparatus is to be installed and operated. For instance, where the sanitizing apparatus is located in the general vicinity of an open door (i.e., entranceway of a grocery store), it may be necessary to provide both a heating and cooling function, as the ambient temperature may be prone to fluctuations.

The construction and arrangement of the features in the various exemplary embodiments is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter herein. For example, elements shown as integrally formed may be constructed of multiple parts or elements, the position of elements may be reversed or otherwise varied, and the nature or number of discrete elements or positions may be altered or varied. Other substitutions, modifications changes and omissions may also be made in design, operating conditions, and arrangement of the various exemplary embodiments without departing from the present scope of the disclosure. It will also be understood that each feature of each embodiment discussed herein, and of each reference cited herein, can be used in combination with the features of any other combination. All patents and publications discussed herein are incorporated by reference herein in their entirety.

What is claimed is:

1. A sanitizing apparatus comprising:
a housing defining an enclosure;
a UV radiation source arranged within the housing, including a UV lamp and configured to provide sterilizing UV radiation to the enclosure; and
a temperature control device associated with the UV radiation source and configured to direct a flow of air into a near field ambient environment of the UV lamp to control a temperature of the UV lamp to within an optimal temperature range.

2. The sanitizing apparatus of claim 1, further comprising a plurality of additional UV radiation sources.

3. The sanitizing apparatus of claim 2, further comprising a plurality of additional temperature control devices, each additional temperature control device being associated with one of the plurality of additional UV radiation sources.

4. The sanitizing apparatus of claim 2, wherein the temperature control device is further configured to direct a plurality of flows of air into a plurality of near field ambient environments of a plurality of additional UV lamps included within the plurality of additional UV radiation sources.

5. The sanitizing apparatus of claim 1, wherein the temperature control device is configured to control temperature to maintain an output of the UV lamp of at least 80% of a maximum output.

6. The sanitizing apparatus of claim 1, wherein the UV lamp is a cycle-start UV-C lamp.

7. The sanitizing apparatus of claim 1, wherein the UV lamp is a continuous UV-C lamp.

8. The sanitizing apparatus of claim 1, wherein the temperature control device further includes at least one temperature sensor and at least one temperature control circuit.

9. The sanitizing apparatus of claim 1, wherein the temperature control device further includes a cooling element.

10. The sanitizing apparatus of claim 1, wherein the temperature control device further includes a heating element.

11. The sanitizing apparatus of claim 1, further comprising a plenum configured to:
receive the flow of air from the temperature control device;
direct the flow of air into the near field ambient environment of the UV lamp to generate sanitized air; and
selectively direct the sanitized air to the enclosure or to an environment surrounding the sanitizing apparatus depending on a selected mode of operation.

12. A method of sanitizing objects, the method comprising:
placing an object to be sterilized in an enclosure defined by a housing of a sanitizing apparatus;
activating a UV radiation source disposed within the housing, the UV radiation source including a UV lamp configured to provide UV radiation to the enclosure;
directing a flow of air into a near field ambient environment of the UV lamp with a temperature control device; and
maintaining a temperature of the UV lamp within an optimal temperature range.

13. The method of claim 12, further comprising activating a plurality of additional UV radiation sources disposed within the housing.

14. The method of claim 13, further comprising directing a plurality of additional flows of air into respective near field ambient environments of the plurality of additional UV radiation sources via a plurality of additional temperature control devices.

15. The method of claim 13, further comprising directing a plurality of additional flows of air into respective near field ambient environments of the plurality of additional UV radiation sources via the temperature control device.

16. The method of claim 12, wherein maintaining the temperature of the UV lamp includes controlling the temperature to maintain an output of the UV lamp of at least 80% of a maximum output.

17. The method of claim 12, further comprising:
measuring the temperature of the near field ambient environment via at least one temperature sensor; and
controlling the temperature control device via at least one temperature control circuit.

18. The method of claim 12, further comprising cooling the flow of air via a cooling element included in the temperature control device.

19. The method of claim 12, further comprising heating the flow of air via a heating element included in the temperature control device.

20. The method of claim 12, further comprising:
receiving the flow of air from the temperature control device with a plenum;
directing the flow of air, by the plenum, into a near field ambient environment of the UV lamp to generate sanitized air; and
selectively directing the sanitized air to the enclosure or to an environment surrounding the sanitizing apparatus depending on a selected mode of operation.

21. The method of claim 12, wherein the UV lamp configured to provide UV light to the enclosure is configured to provide UV-C radiation.
